(19) United States
(12) Patent Application Publication
(10) Pub. No.: US 2018/0271658 A1
(43) Pub. Date: Sep. 27, 2018

(54) RENOVATED TITANIUM MESH

(71) Applicant: Beijing Chunlizhengda Medical Instruments Co., Ltd., Beijing (CN)

(72) Inventors: Chunbao SHI, Beijing (CN); Huiguang ZHONG, Beijing (CN); Fengbao XIE, Beijing (CN); Yankai XU, Beijing (CN); Xiangxiang XU, Beijing (CN)

(21) Appl. No.: 15/990,731

(22) Filed: May 28, 2018

Related U.S. Application Data
(63) Continuation of application No. PCT/CN2016/097826, filed on Sep. 1, 2016.

(30) Foreign Application Priority Data
Dec. 17, 2015 (CN) 201510952813.5

(51) Int. Cl. A61F 2/30

(52) U.S. Cl. CPC A61F 2/30734; A61F 2/30771; A61F 2310/00023; A61F 2/38; A61F 2310/00359; A61F 2002/3093; A61F 2002/30736

(57) ABSTRACT

The present invention discloses a renovated titanium mesh used for repairing bone defects in knee joint replacement. The renovated titanium mesh (1) includes transverse ribs (2) for implanting the renovated titanium mesh into a human body and longitudinal ribs (3) for supporting the transverse ribs (2). The transverse ribs (2) and the longitudinal ribs (3) are crisscrossed to form the meshed renovated titanium mesh (1). In the renovated titanium mesh of the present invention, the meshed structure of the renovated titanium mesh can ensure the bone ingrowth of a patient, and the renovated titanium mesh can be cut to provide a high degree of matching for the patient and to thus satisfy the patient's demand; the renovated titanium mesh can better ensure the bone ingrowth of the patient and be advantageous for the later recovery of the patient.

### FIELD OF THE INVENTION

The present invention relates to a medical prosthesis, and in particular to a renovated titanium mesh capable of repairing bone defects in knee joint replacements.

### BACKGROUND OF THE INVENTION

As an implantable prosthesis in knee joint replacements, a renovated titanium mesh mainly functions to remedy bone defects of a patient. In the prior art, renovated cushions are mainly used in bone defect prosthetics in China and foreign countries. The renovated cushions mainly consist of solid structures in various shapes to adapt to different done defect patients, and the renovated cushions are mainly applied in revision surgeries and primary surgical replacements for severe bone defects. With the increase of revision cases in the clinical surgeries in China, the application of renovated cushions is increasing, and some disadvantages of the renovated cushions are found.

At present, in the Chinese market, renovated metal cushions are mainly used for repairing bone defects in knee joint replacements. The metal cushions are mainly of a solid structure made of titanium alloy or a 3D printed trabecular structure. A screw hole is additionally formed in a middle portion of each metal cushion to fix this metal cushion to a knee prosthesis. However, this structure is heavy and disadvantageous for bone ingrowth, so that it is disadvantage for the patient's recovery. Moreover, since the metal cushions are fixed in specification, the demand of a patient with severe bone defects cannot be satisfied.

The currently used renovated titanium meshes have the following problems: the renovated titanium meshes are of a meshed structure which is less stable than the solid structure and easy to break. The meshed structure has a relatively centralized stress and has higher requirements on the strength of a support pillar. Additionally, it is difficult to produce titanium meshes and it is disadvantageous for batch production.

### SUMMARY OF THE INVENTION

An objective of the present invention is to provide a renovated titanium mesh capable of repairing bone defects in knee joint replacements.

For this purpose, the renovated titanium mesh of the present invention employs the following specific technical solutions.

A renovated titanium mesh used for repairing bone defects in knee joint replacements is provided, wherein the renovated titanium mesh includes transverse ribs for implanting the renovated titanium mesh into a human body and longitudinal ribs for supporting the transverse ribs; and, the transverse ribs and the longitudinal ribs are crisscrossed to form the meshed renovated titanium mesh.

Further, a plurality of transverse ribs and a plurality of longitudinal ribs are provided in the renovated titanium mesh.

Further, there is a hollow portion between the transverse ribs and the longitudinal ribs, and an allogeneic bone and/or a knee joint prosthesis outside the renovated titanium mesh can enter into the hollow portion and be then fixed with the renovated titanium mesh.

Further, the renovated titanium mesh is enclosed to form an annular shape, and the cross-section of the renovated titanium mesh is a wedged shape having a wider top and a narrower bottom.

Further, the renovated titanium mesh is made of titanium alloy material.

Further, there is an included angle of 65° to 75° between a side wall and a bottom wall of each of the transverse ribs, and the renovated titanium mesh can be stably implanted in the bone by inserting the transverse ribs into the bone at an included angle.

The renovated titanium mesh of the present invention has the following advantages:

1) the meshed structure of the renovated titanium mesh can ensure the bone ingrowth of a patient, and the renovated titanium mesh can be cut to provide a high degree of matching for the patient and to thus satisfy the patient's demand;

2) the renovated titanium mesh can better ensure the bone ingrowth of the patient and be advantageous for the later recovery of the patient;

3) since the renovated titanium mesh is used together with few instruments, the operation duration is saved;

4) since the use of interventional material is reduced, the weight is reduced, so that it is advantageous for repairing a bone defect part, instead of purely replacing the bone defect part with prosthesis; and

5) the renovated titanium mesh can cope with various special cases, and is advantageous for the smooth progress of the operation.

### BRIEF DESCRIPTION OF THE DRAWINGS

FIG. 1 is a schematic structure diagram of a renovated titanium mesh of the present invention.

### REFERENCE NUMERALS

1: renovated titanium mesh; 2: transverse rib; 3: longitudinal rib; and, 4: hollow portion.

### DETAILED DESCRIPTION OF THE INVENTION

To better explain the objectives, structures and functions of the present invention, the renovated titanium mesh of the present invention will be further described below in detail with reference to the accompanying drawings.

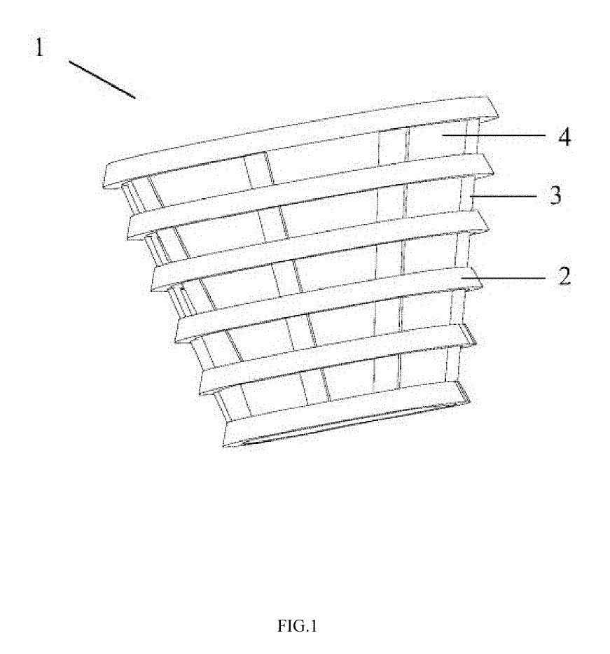

As shown in FIG. 1, the renovated titanium mesh of the present invention is shown, which is used for repairing bone defects in knee joint replacements. The renovated titanium mesh includes a plurality of transverse ribs 2 and a plurality of longitudinal ribs 3. By inserting the transverse ribs 2 into the bone, the renovated titanium mesh 1 is implanted into the human body. The transverse ribs 2 and the longitudinal ribs 3 are crisscrossed to form the meshed renovated titanium mesh 1, and the longitudinal ribs 3 are used for supporting the transverse ribs 2 so as to support the whole structure of the renovated titanium mesh 1.

Further, there is a hollow portion 4 between the transverse ribs 2 and the longitudinal ribs 3, and an allogeneic bone and/or a knee joint prosthesis outside the renovated titanium mesh 1 can enter into the hollow portion and be then fixed to the renovated titanium mesh. By using with a platform support or femoral condyle, the renovated titanium mesh may be fixed to the human body.

Further, the renovated titanium mesh 1 is enclosed to form an annular shape, and the cross-section of the renovated titanium mesh 1 is a wedged shape having a wider top and a narrower bottom, so that a stable interior structure may be realized. In addition, the shape of the renovated titanium mesh 1 is designed by simulating the bone of the human body, and the renovated titanium mesh 1 may be cut into various sizes, so that a higher degree of matching is provided for patients, and the operation requirements of different patients are satisfied. With the meshed structure of the renovated titanium mesh 1, the use of interventional material is reduced, and the weight is also reduced. Moreover, since the renovated titanium mesh is used together with few instruments, the operation duration is saved.

Further, the renovated titanium mesh 1 of the present invention is made of titanium alloy material; and, there is an included angle of 65° to 75° between a side wall and a bottom wall of each of the transverse ribs 2, that is, there is an included angle of 15° to 25° between a side wall of each of the transverse ribs 2 and an axial direction. By inserting the transverse ribs into the bone at an included angle, the renovated titanium mesh may be stably implanted in the bone.

In the renovated titanium mesh of the present invention, the meshed structure of the renovated titanium mesh can ensure the bone ingrowth of a patient, and the renovated titanium mesh can be cut to provide a high degree of matching for the patient and to thus satisfy the patient's demand; the renovated titanium mesh can better ensure the bone ingrowth of the patient and be advantageous for the later recovery of the patient; since the renovated titanium mesh is used together with few instruments, the operation duration is saved; since the use of interventional material is reduced, the weight is reduced, so that it is advantageous for repairing a bone defect part, instead of purely replacing the bone defect part with prosthesis; and, the renovated titanium mesh can cope with various special cases, and is advantageous for the smooth progress of the operation.

Although the present invention has been further described above by specific embodiments, it should be understood that the detailed description shall not be interpreted as limitations to the essence and scope of the present invention. Various modifications made to the embodiments by a person of ordinary skill in the art after reading the specification shall fall into the protection scope of the present invention.

What is claimed is:

1. A renovated titanium mesh for repairing bone defects in knee joint replacement surgery, wherein the renovated titanium mesh comprises at least one transverse rib for implanting the renovated titanium mesh into a human body and at least one longitudinal rib for supporting the transverse rib; the transverse rib and the longitudinal rib are crisscrossed and combined to form a meshed renovated titanium mesh; and, the renovated titanium mesh is enclosed to form an annular shape; and
a cross-section of the renovated titanium mesh is a wedged shape having a wider top and a narrower bottom.

2. The renovated titanium mesh according to claim 1, wherein a plurality of transverse ribs and a plurality of longitudinal ribs are provided in the renovated titanium mesh.

3. The renovated titanium mesh according to claim 1, wherein a hollow portion is provided between the transverse rib and the longitudinal rib; and
an allogeneic bone and/or a knee joint prosthesis outside the renovated titanium mesh enter into the hollow portion and being fixed to the renovated titanium mesh.

4. The renovated titanium mesh according to claim 1, wherein the renovated titanium mesh is made of titanium alloy material.

5. The renovated titanium mesh according to claim 1, wherein an included angle of 65° to 75° is formed between a side wall and a bottom wall of the transverse rib; and the renovated titanium mesh is stably implanted into the bone by inserting the transverse rib into the bone with the included angle.
